(19) United States
(12) Patent Application Publication
(10) Pub. No.: US 2003/0148411 A1
(43) Pub. Date: Aug. 7, 2003

(54) Method for detecting helicobacter pylori and heilmanii in fecal and salivary specimen and biopsy material

(76) Inventors: Franz Paul Armbruster, Bensheim (DE); Katarina Crevar, Babenhausen (DE); Jana Ruppert, Lautertal (DE)

Correspondence Address: Jesse A Hirshman, Kirkpatrick & Lockhart, Henry W Oliver Building, 535 Smithfield Street, Pittsburgh, PA, 15222-2312, US

(21) Appl. No.: 10/203,679

(22) PCT Filed: Feb. 14, 2001
(86) PCT No.: PCT/EP01/01639

(30) Foreign Application Priority Data
Feb. 14, 2000 (DE) 10006432.9

(51) Int. Cl. G01N 33/554; G01N 33/569

(52) U.S. Cl. 435/7.32

(57) ABSTRACT

The invention relates to a method for detecting pathogenic organisms, in particular Helicobacter pylori and H.heilmanii, in fecal, salivary and secretory samples by means of a double-antibody sandwich assay. The inventive method is characterized by dissolving or dispersing the sample having the pathogen antigen in a buffer solution and contacting the buffer solution with a solid phase to which at least two primary antibodies are bound, one of which specifically binds to the pathogenic antigen and the other to human immunoglobulin A; washing the solid phase of non-specifically bound proteins and contacting the solid phase with a secondary antibody which specifically binds to pathogenic antigen, and determining the quantity of specifically bound secondary antibodies.

The invention relates to a method of detecting Helicobacter antigens in fecal and salivary samples or biopsy materials.

The human gastric mucosa is often colonised by bacteria of the genera Helicobacter pylon and heilmanii or Campylobacter. Infection with these bacteria probably occurs from person to person, but drinking water and foodstuffs are not excluded as sources of infection. The strain Helicobacter heilmanii is passed on by domestic animals such as cats, dogs, rabbits, and also by farm animals such as cows. For this reason persons involved in farming and animal care are particular subjected to infection. In Germany, about 10% of school children, 30% of people in their thirties and about 75% of senior citizens are infected with H.pylori. World-wide, ca. 50% of people carry this infection. 80% of incidents of gastritis, 95% of duodenal ulcers and 70% of ventricular ulcers are caused by H.pylori. Further, chronic H.pylori gastritis (Type B gastritis) is considered as a precursor for gastro-adeno carcinoma and gastric lymphoma. According to the WHO, H.pylori is a carcinogen of the highest cancer risk class. Only a small proportion of H.pylori infected people develop symptoms. Many live, despite a gastritis, without noticeable complaints or they attribute the rather non-specific symptoms to other causes. An acute H.pylori gastritis manifests itself through indeterminate pains in the upper and middle stomach, feelings of pressure and fullness, acid eructation, heartburn and through nausea and retching. In practically all patients having a Type B gastritis, an H.pylori infection can be found. Despite the commonly massive immune reaction the infection becomes chronic with these patients. Whether an ulcer develops depends upon the immune system of the patient and upon the aggressiveness of the bacteria or the kind of bacteria type. Spontaneous recoveries are rare. As a rule, a H.pylori infection, if not treated, persists for the whole life.

An H.pylori infection can be diagnosed by means of culturing the pathogen from an antrum or corpus biopsy or by means of histological testing of the tissue. Further diagnostic methods are the CLO test (test for Campylobacter-Like Organisms) or the urease urea test, the ¹³C-Isotope breath test, the detection of antibodies against H.pyloriin the serum, the PCR detection of Helicobacter DNA in a gastric fluid or fecal sample and the detection of H.pylori antigens in a fecal sample. U.S. Pat. No. 5,716,791 (Larka et al.) and EP 0 806 667 (Meridian Diagnostics Inc.) describe an immunoassay for H.pylori antigens in the stool. The assay is based on two affinity purified polyclonal antibodies against H.pylori antigen. Further there is available from Connex GmbH, Martinsried, Germany, an instant test which is based on a lateral flow chromatography of gold-marked monoclonal antibodies against H.pylori stool antigens. These so-called HpSA tests (Helicobacter pylori Stool Antigen) have provided in various clinical studies a good agreement with cases diagnosed by other means, but a high percentage of the tests do not lead to an unambiguous result. The HpSA test is, moreover, unsuitable for monitoring an eradication treatment, since it only functions with abundant quantities of H.pylori antigen in the stool. The further diagnostic methods are in part very complicated, stressful for the patient, or too expensive for routine testing. The serological methods are disadvantageous in that they do not permit a course of treatment to be monitored, since the antibody titre remains high even months after an eradication of the bacteria. A monitoring of the course of treatment is, however, essential since resistance may be present against the antibiotics, such as Clarithromycin, Metronidazole, Amoxicillin, Omeprazol or Proton Pump Inhibitor, used. Treatment of the infections by means of antibiotics and alternative natural remedies thus requires a simple, reliable and sensitive test method for H.pylori.

This object is achieved by means of an immunoassay in accordance to claim 1. Preferred embodiments of the invention are indicated in the sub-claims.

The method in accordance with the invention for detecting Helicobacter pathogen antigens in a sample is characterised by the taking up and suspension in a sample buffer of a human sample to be tested for the pathogen; the bringing together of the sample buffer having the pathogen antigen with a solid phase, to which at least two different primary antibodies are bound, of which one is capable of binding antigens of the pathogen and the other is capable of binding human immunoglobulin; washing of the solid phase of not specifically bound antigens; bringing together of the solid phase with a secondary antibody which specifically recognises antigens of the pathogen, and determination of the quantity of bound secondary antibodies.

The quantity of the secondary antibody bound to the solid phase can be determined for example by means of a marking of the antibody. The marker may be radioactive, a luminescent or fluorescent group, a group such as biotin, which can be bound by a further molecule such as streptavidin, or an enzyme such as peroxidase, which catalyses a detection reaction. The quantity of bound secondary antibody can also be determined by means of a further antibody which specifically recognises the type of the secondary antibody and carries one of the above-mentioned markers.

The second primary antibody against human immunoglobulin, bound to the solid phase, is preferably an antibody which binds Human-IgA and particularly preferably binds secretory Human-IgA, which is secreted into the saliva and in the large intestine. The designation secretory Human-IgA here includes Human-IgA and if applicable also the so-called secretory components (MW: 70 kDa) which are produced by the epithelial cells for transport and for protection of the IgA from proteolytic enzymes. Particularly preferred are antibodies against IgA2, since Helicobacter and also other microorganisms create proteases which can fragment sIgA1. Very particularly preferred is a mixture of two primary antibodies which specifically bind IgA1 and IgA2.

In a preferred embodiment of the invention the first primary antibody, bound to the solid phase, is a polyclonal rabbit-anti-H.pylori antibody. The second primary antibody, bound the solid phase, is a polyclonal goat-anti-Human-sIgA antibody tested for cross-reactivity. The secondary antibody is biotin-conjugated rabbit-anti-H.pylori antibody, so that the quantity of the bound secondary antibody can be determined through the binding of peroxidase-conjugated streptavidin and colour reaction with tetramethyl-benzidin.

In a second preferred embodiment the first primary antibody, bound to the solid phase, is a rabbit-anti-H.pylori antibody, the second primary antibody is a rabbit-anti-human-sIgA antibody and the secondary antibody is a polyclonal horseradish peroxidase-conjugated goat-anti-H.pylori antibody. There can, however, also be employed other immunoglobulins of the horse, cattle, pig, sheep, goat, rabbit, guinea pig, rat, mouse or another animal. It is in particular to be ensured that the second primary antibody for human immunoglobulin does not bind the secondary antibody against the pathogen antigen in the salivary or fecal sample. The antibodies may, in principle, be monoclonal. In particular, the second primary antibody may be a monoclonal antibody for Human-IgA2. It is considered better to employ a plurality of a monoclonal antibodies which recognise different epitopes of the pathogen or of human immunoglobulin. With monoclonal primary antibodies it is considered better to employ a mixture of a plurality monoclonal antibodies, in order to broaden the specificity of the assay.

The secondary antibodies are preferably conjugated with one or more markers such as biotin, fluorescene, ruthenium, europium, gold particles, alkaline phosphatase, galactosidase, or horseradish peroxidase. However, other suitable marker or detection systems can also be employed.

### TEST PRINCIPLE EXEMPLIFIED BY AN ELISA

The ELISA (enzyme-linked immunosorbent assay) in accordance with the invention serves for the qualitative and quantitative detection of H.pylori antigens in fecal and salivary samples and biopsy material. The H.pylori antigen is, in a first step, released from the sample and bound by a preferably polyclonal anti-H.pylori antibody, which in conventional manner is bound to a microtitre plate or some other solid phase. There is further fixed to the solid phase a second primary antibody, which recognizes human immunogloblulin and preferably human secretory IgA. Since H.pylori antigens in a fecal or salivary sample have been in contact with the body's own human immune system, some or all H.pylori epitopes are already bound by immunoglobulins and are no longer accessible for bonding by means of the first primary antibody against the pathogen. Despite this however, the pathogen antigens in the fecal or salivary sample and biopsy material are, even if an immune reaction against these is already present, concentrated and bound to the solid phase by the second primary antibody against human immunoglobulin. Thus, on the one hand H.pylori antigens are directly bound and on the other hand are indirectly bound via their binding to secreted human immunoglobulin in the saliva or intestinal tract. Then, in the second step, bound H.pylori antigen is then directly detected by means of anti-H.pylori secondary antibody. By means of a suitable system the quantity of bound secondary antibody is then quantified.

Through the high specificity of the secondary antibody against H.pylori antigen, false positive results are excluded. The overall specificity of a double antibody assay is, namely, in substance determined by the specificity of the secondary antibody. The fact that in the first binding step human secretory antibody with pathogen antigens is also bound increases in the case of H.pylori not only the sensitivity but also the bandwidth of the test. It is particularly surprising that clearly in a considerable number of cases all immunogenic epitopes of H.pylori are recognized by the human immune reaction present. Clearly, in the case of Helicobacter organisms the number immunogenic epitopes is limited, so that the pathogen epitopes recognized by the human immune system and those recognised by the animal primary and secondary antibodies of the double-antibody binding assay are often identical. This applies in particular for a H.pylori colonization of the mouth and for the later phase of an eradication treatment, in which the human immune reaction disguises the now reduced quantity of H.pylori epitopes which are still present in the fecal or salivary sample. By means of the test principle in accordance to the invention, small and even masked quantities of H.pylori antigen can be detected.

The occurrence of Helicobacter pylori in the saliva has to date been a subject only of academic interest with regard to this suspected mode of transmission (Namavar F. et aI ., Eur. J. Clin. Microbiol. Infect. Dis., 1995, 14(3), pp. 234-7; Shimada T. et al., Lancet, 1994, 343(8913), pp. 1636-7). By means of the method in accordance with the invention, the lower limit for detection of H.pylori is now brought so low that even a test of the saliva permits diagnosis of an H.pylori infection. In particular for monitoring a course of treatment it is important to test the saliva of the patient for H.pylori antigens. The experiments carried out by the applicants provided indications that in the mouth—probably beneath metal fillings and dental prostheses, etc.—focuses of infection with H.pylori can be found which can survive the conventional medical eradication treatment and after conclusion of the treatment lead to renewed infection of the gastric mucosa. An eradication treatment can thus be considered be concluded only when no H.pylori antigens can be detected either in the fecal or in the salivary sample.

The sandwich test principle in accordance with the invention with a second primary antibody against human immunoglobulin, in particular against human secretory IgA, is not restricted to employment on a microtitre plate. It can be adapted for fully automated processes which work with coated beads or particles. The principle in accordance with the invention, with a second primary antibody against secretory human-IgA, is suitable in general for the diagnosis of pathogens which occur in the mouth or the intestinal tract and bring about a massive immune reaction.

In general it is preferred if the first primary antibody is a pool polyclonal antibody against various H.pylori types. This applies also for the secondary antibody, since this improves the reliability of the test.

The invention and its advantages will now be described with reference to preferred embodiments and examples and with reference to accompanying drawings. There is shown:

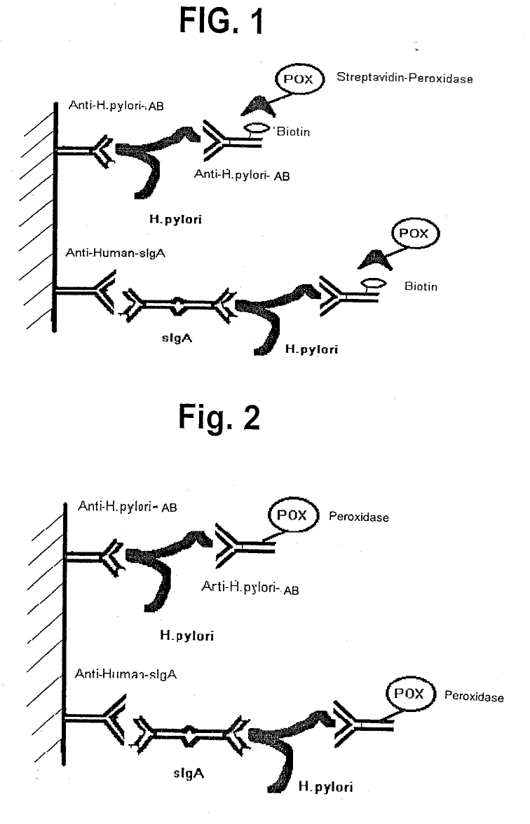

FIG. 1 a schematic of the principle of the immunoassay for H.pylori antigen in accordance with the invention;

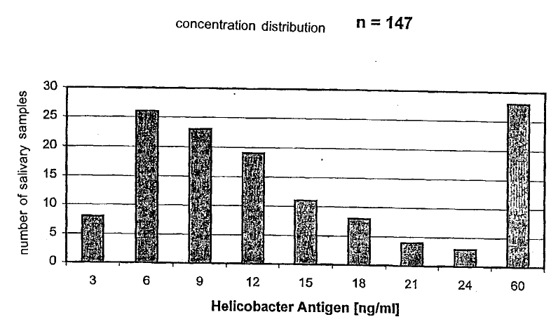

FIG. 2 a further exemplary form of the immunoassay in accordance with the invention;

FIG. 3 a graphical illustration of the H.pylori antigen concentration in saliva determined in accordance with the invention before and after an eradication treatment.

### EXAMPLE 1 —Detection of H.pylori antigen in stool and saliva

Preparation of the fecal sample: ca. 100 mg stool was weighed and dispersed at room temperature in 5 ml PBS washing buffer having 0.1% Triton X-100 (8 mM Na₂HPO₄, 15 mM M K₂H₂PO₄, 3 mM KCI, 12.5 mM NaCI, 0.1% Triton X-100, 0.02% Thimerosal, pH 7.4). The dosing and homogenisation of the fecal sample was effected preferably with a sample preparation system of Roche Diagnostik, Mannheim, Germany (Order No. 745804). The homogenate was centrifuged in a desk centrifuge for 10 minutes at 3000 rpm, 1 ml product was transferred to an Eppendorf vial and centrifuged for 5 minutes at 13000 rpm in an Eppendorf centrifuge. The product was directly employed in the ELISA test.

Preparation of the salivary sample: The salivary sample was diluted 1:4 or 1:5, depending on viscosity, in assay buffer and directly employed in the binding assay. In the case of salivary samples it is advantageous if the assay buffer contains a protease inhibitor such as, for example, 5 mM PMSF. There can also be employed commercial protease inhibitors such as Pefabloc SC (Roche Diagnostics).

Coating of the microtitre plate: The wells of the microtitre plate were coated with polyclonal antibodies against H.pylori antigen and human immunoglobulin-A. For this purpose there was dosed into each well 100 μg commercially available polyclonal rabbit-anti -H.pylori antibody (DAKO, Hamburg) dissolved in 200 μl60 mM NaCO₃, pH 9.6 , and the plate incubated overnight at 4° C. The rabbit-anti-H.pylori antibody solution in the wells was removed and each well washed with 200 μl washing buffer (PBS, pH 7.4 with 0.1% Triton X-100). There was then dosed into each well 100 g rabbit-anti-human-sIgA antibody (DAKO, Hamburg) dissolved in 200 μl 60 mM NaCO₃, pH 9.6 , and the plate incubated for 1 hour at room temperature. The anti-human-IgA antibody solution in the wells was removed and each well again Washed five times each with 125 μl washing buffer. After the last washing procedure the wells of the microtitre plate were knocked out onto absorbent paper.

Binding assay: The tests were all carried out in duplicate. 100 μl standard and sample were pipetted in duplicate into the antibody coated wells of the microtitre plate and incubated at room temperature for 1 hour while been shaken. The solutions were removed and the wells of the plate washed five times in each case with 250 μl washing buffer. After the last washing procedure the microtitre plate was knocked out dry on absorbent paper.

Detection of binding: Into the wells there was dosed in each case 100 μl biotin-conjugated polyclonal rabbit-anti-H.pylori antibody (1:10000; DAKO, Hamburg) or horseradish peroxidase (HRP)-conjugated polyclonal goat-anti-H.pylori antibody (KPL, Kirkegaard and Perry Laboratories, Gaithersburg, MD; a mixture of polyclonal antibodies against the H.pylori types ATCC 43504, 43526, 43579) diluted 1:1000 in washing buffer, and incubated at room temperature for 1 hour while been vibrated. The solution was removed from the wells and each well washed five times with 200 μl washing buffer.

Quantitative determination: For the colour reaction, in the case of horseradish peroxidase-conjugated goat-anti-H.pylori antibody, 100 μl Tetramethylbenzidin (TMB)-substrate solution (ready-for-use, from NOVUM Diagnostika GmbH, Dietzenbach, Germany) was dosed into the wells and after about 20 minutes the colour development was stopped by the addition of 50 μl 1 0.4 M H₂SO₄. In the case of biotin-coupled rabbit-anti -H.pylori antibody, 100 μl horseradish peroxidase-conjugated streptavidin (DAKO) was coated on, 1:10000 diluted in washing buffer, and incubated for 1 hour at room temperature while being vibrated, washed five times with washing buffer, and only then was the chromogenic substance added. The colour development was in each case determined by measurement of extinction (optical density) at 450 nm. The following Tables 1 and 2 show representative results from healthy and H.pylori-infected patients, whereby the determinations in accordance with the invention were repeated on different days. The determinations were effected as indicated with different detection systems and/or secondary antibodies.

### COMPARATIVE EXAMPLE 2

Immunoassay without second antibody against Human-immunoglobulin-A

The preparation of the fecal and salivary samples and of the binding assay were effected exactly as in Example 1, except that the wells of the microtitre plate were coated exclusively with affinity purified polyclonal rabbit-immunoglobulin against Helicobacter pylori or with anti-human-sIgA antibodies. All washing, coating and binding steps, and the colour development were effected in parallel manner on the same microtitre plate.

TABLE 1
Extinction (Optical Density-OD) at 450 nm¹

| | microtitre plates | | | | | | | |
|---|---|---|---|---|---|---|---|---|
| | Coating | | | | | | | |
| | Primary antibody | | | | | | | |
| | Polyclonal Rabbit | | | | | | | |
| | Anti-H. pylori-AB + | | | Comparison | Comparison | | Comparison | Comaparison |
| | Polyclonal Rabbit | | | without Anti- | only Anti- | AntiH.pylori + | without Anti- | only Anti- |
| | Anti-Human-slgA-AB | | | Human-slgA | Human-slgA | Anti-Human-slgA | Human-slgA | Human-slgA |
| | Secondary antibody | | | | | | | |
| | Biotin-conjugated Rabbit-Anti-H.pylori-AB | | | | | HRP-conjugated Goat-Anti-H.pylori-AB | | |
| Measurement/Date | 12.01.00 | 14.01.00 | 25.01.00 | 25.01.00 | 25.01.00 | 25.01.00 | 25.01.00 | 25.01.00 |
| Pat T. | 0,400 | 0,458 | 0,461 | 0,436 | 0,144 | 0,258 | 0,170 | 0,181 |
| Nr. 15294 | 2,050 | 1,635 | n.g | n.g | n.g | n.g | n.g | n.g |
| Pat: Car | 0,925 | 0,654 | 0,505 | 0,300 | 0,313 | 0,720 | 0,210 | 0,690 |
| Pat. Woz | 0,258 | 0,270 | 0,162 | 0,101 | 0,117 | 0,233 | 0,085 | 0,216 |
| Pat. K | — | | 0,377 | 0,141 | 0,284 | 0,538 | 0,112 | 0,449 |
| Pat. Tas | | | 1,798 | 1,768 | 0,107 | 0,580 | 0,447 | 0,182 |
| Pat. Hal | | | 0,222 | 0,263 | 0,199 | 0,189 | 0,121 | 0,183 |
| Pat. Hof | | | 1,023 | 0,831 | 0,315 | 0,463 | 0,163 | 0,398 |
| Non-specific | 0,150 | 0,219 | 0,228 | 0,211 | 0,154 | 0,200 | 0,157 | 0,187 |
| Binding | | | | | | | | |
| (Standard buffer) | | | | | | | | |

Ad1: All extinction values are mean values of two measurements

TABLE 2
Measurement values (non-specific binding deducted)

| | microtitre plates | | | | | | | |
|---|---|---|---|---|---|---|---|---|
| | Coating | | | | | | | |
| | Primary antibody | | | | | | | |
| | Polyclonal Rabbit | | | | | | | |
| | Anti-H.pylori + | | | Comparison | Comparison | | Comparison | Comparison |
| | Polyclonal Rabbit Anti- | | | without Anti- | only Anti- | Anti-H.pylori + | without Anti- | only Anti- |
| | Humans-slgA | | | Human-slgA | Humans-slgA | Anti-Human-slgA | Human-slgA | Human-slgA |
| | Secondary antibody | | | | | | | |
| | Biotin-conjugated Rabbit-Anti-H.pylori-AB | | | | | HRP-conjugated Goat-Anti-H.pylori-AB | | |
| Measurement | 12.01.00 | 14.01.00 | 25.01.00 | 25.01.00 | 25.01.00 | 25.01.00 | 25.01.00 | 25.01.00 |
| date | | | | | | | | |
| Patient T. | 0,250 | 0,239 | 0,233 | 0,225 | −0,010 | 0,058 | 0,013 | −0,006 |
| Pat. Nr. 15294 | 1,900 | 1,635 | n.g. | n.g. | n.g. | n.g. | n.g. | n.g. |
| Pat: Car | 0,775 | 0,654 | 0,277 | 0,089 | 0,159 | 0,520 | 0,053 | 0,503 |
| Pat. Woz | 0,108 | 0,270 | −0,066 | −0,110 | −0,037 | 0,033 | −0,072 | 0,029 |
| Pat. K | — | — | 0,149 | −0,070 | 0,130 | 0,338 | −0,045 | 0,262 |
| Pat. Tas | | | 1,570 | 1,557 | −0,047 | 0,380 | 0,290 | −0,005 |
| Pat. Hal | | | −0,006 | 0,052 | 0,045 | −0,011 | −0,036 | −0,004 |
| Pat. Hof | | | 0,795 | 0,620 | 0,161 | 0,263 | 0,006 | 0,211 |

### DISCUSSION

Tables 1 and 2 show that in some patients the H.pylori antigens present in the fecal sample were completely masked by the body's own immune system and thus could not be bound by the analytic primary antibody against the pathogens. In the case of such masking of the antigens, a conventional double-antibody immunoassay leads to the false result that no H.pylori infection is present or is no longer present. With the method in accordance with the invention, in contrast, H.pylori antigens already bound or masked by the human immune system were bound to the solid phase (see Table 2, the field with the gray background) by the second anti-human-sIgA-primary antibody. The masking effect was not dependent upon the secondary antibody used nor dependent upon whether the sample was a salivary or fecal sample. Further, the results make it clear that the primary bindings of H.pylori to the solid phase via anti-human-sIgA antibodies and anti-H.pylori antibodies are mutually exclusive in many cases, so that clearly an either/or situation was present with regard to the binding. The possible primary binding of the H.pylori antigens to the solid phase via the binding to human immunoglobulin-A is thus an essential feature of the diagnosis in accordance with the invention.

### EXAMPLE 3—SALIVA MASS SCREENING

Salivary samples were taken from 150 patients suspected of H.pylori infection or after completed eradication treatment, and were tested for the presence of H.pylori antigens. The analysis was effected as indicated in Example 1 with the exception that the assay buffer additionally contained a protease inhibitor. The detection limit for Helicobacter antigen in saliva lies in the above-described test (with HRP-conjugated goat-anti-H.pylori-antibody and tetramethylbenzidin) at about 2 pg H.pylori -antigen per millilitre assay buffer. By optimization of the second antibody against the pathogen antigen, and selection of a luminescence detection system, it should be possible to reach a detection limit of 0.2 pg per millimetre, and below.

The result of the mass screening is shown in FIG. 3 in a bar chart. The bar chart shows that patients with an H.pylori infection present in the saliva as a rule have more than 60 ng/ml H.pylori antigens. The testing of the saliva is thus suitable for the detection of the presence of an H.pylori infection. H.pylori antigen concentrations below 25 ng per ml saliva indicate, in contrast, a non-specific cross-reaction with other pathogens.

Thus, for the first time, it has been proven that an H.pylori infection can be detected by means of an immunological saliva test which is relatively simple to carry out. Thus, the success of an eradication treatment can be relatively simply monitored. The previous analyses of saliva for H.pylori in contrast permitted no diagnosis of a H.pylori infection of the intestinal tract, but were based on a academic investigation of a possible mode of transmission.

1. Method of detecting pathogenic organisms in faecal, salivary and secretory samples by means of a double-antibody sandwich-binding assay, characterised by
taking up or dispersing a sample having pathogen antigen in a buffer solution and bringing together the buffer solution with a solid phase to which at least two primary antibodies are bound, of which one specifically binds a pathogen antigen and the other specifically binds human immunoglobulin-A;
washing the solid phase of not specifically bound proteins and bringing together the solid phase with a secondary antibody which specifically binds to pathogen antigen, and determining the quantity of specifically bound secondary antibody.

2. Method according to claim 1, wherein the second primary antibody binds secretory human-IgA.

3. Method according to claim 2, wherein the second primary antibody binds secretory human-IgA2.

4. Method according to any of claims 1 to 3, wherein the secondary antibody is conjugated with a group selected from biotin, fluorescene, ruthenium, europium, gold particles, alkaline phosphatase, galactosidase and horseradish peroxidase.

5. Method according to any of claims 1 to 4, wherein the first primary antibody and the secondary antibody bind Helicobacter pylori antigens.

6. Method according to any of claims 1 to 5, wherein the first primary antibody is a mixture of polyclonal and/or monoclonal antibodies against various Helicobacter pylori types.

7. Method according to any of claims 1 to 6, wherein the secondary antibody is a mixture of polyclonal and/or monoclonal antibodies against various Helicobacter pylori types.

8. Method according to any of claims 1 to 4, characterized in that the pathogen is Campylobacter.

9. Method according to any of claims 1 to 4, characterized in that the pathogen is Helicobacter heilmanii.

10. Method according to any of claims 1 to 4, characterized in that the pathogen brings about a massive immune reaction in the human body.

11. Method according to any of preceding claims 1 to 10 for the diagnosis of remaining pathogens in the mouth after a medical eradication treatment.
